(19) United States
(12) Patent Application Publication
(10) Pub. No.: US 2008/0103505 A1
(43) Pub. Date: May 1, 2008

(54) CONTAINMENT DEVICE FOR SITE-SPECIFIC DELIVERY OF A THERAPEUTIC MATERIAL AND METHODS OF USE

(76) Inventor: Hendrik Raoul Andre Fransen, Lochristi (BE)

Correspondence Address: BRINKS HOFER GILSON & LIONE/CHICAGO/COOK, PO BOX 10395, CHICAGO, IL, 60610, US

(21) Appl. No.: 11/867,429

(22) Filed: Oct. 4, 2007

Related U.S. Application Data
(60) Provisional application No. 60/854,563, filed on Oct. 26, 2006.

(51) Int. Cl. A61B 17/58

(52) U.S. Cl. 606/92

(57) ABSTRACT

The present invention is directed to devices and methods for the controlled delivery of a therapeutic material into the treatment site to prevent unintentional migration of the therapeutic material from the treatment site. The devices include an expandable containment body made of a naturally-derived collagenous material and a delivery device to convey the containment body into a treatment location within a body. The invention is also directed to a method of treating a bone, an aneurysm, or arteriovenous malformations by providing the containment device.

### RELATED APPLICATIONS

The present patent document claims the benefit of the filing date under 35 U.S.C. §119(e) of Provisional U.S. Patent Application Ser. No. 60/854,563, filed Oct. 26, 2006, which is hereby incorporated by reference.

### BACKGROUND

1. Technical Field

The present invention generally relates to devices for delivering therapeutic material to a localized area of a patient's body without unintentional leakage into adjacent anatomy.

2. Background Information

Numerous bone conditions or spinal injury can cause painful collapse of vertebral bodies, including osteopenia (osteoporosis), vertebral hemangiomas, multiple myeloma, necorotic lesions (Kummel's Disease, Avascular Necrosis), metastatic disease and complications from steroid and non-steroidal anti-inflammatory drug (NSAID) use. Osteoporosis is a systemic, progressive and chronic disease that is usually characterized by low bone mineral density, deterioration of bony architecture, and reduced overall bone strength. Vertebral body compression fractures (VCF) are more common in people who suffer from these medical indications, often resulting in pain, compromises to activities of daily living, and even prolonged disability. Likewise, degenerative and injured spinal disk rehabilitation (pharmacological or gene therapeutic) protocols to delay the progressions of intradiscal diseases, or even to restore disk health and disk functions, are a part of contemporary research developments and emerging standards of care.

The science of spinal intervention has made great strides in recent years. On some occasions, spinal or poly-trauma patients experience VCFs that may be repaired by vertebroplasty and other spinal reconstructive means. Vertebroplasty, which literally means fixing the vertebral body, has been used in the United States since the mid-1990s to treat pain and progressive deterioration associated with VCF. Most often in this vertebroplasty procedure, bone cement, like opacified polymethyhnethacrylate (PMMA), or other suitable biomaterial alternatives or combinations, is injected percutaneously into the bony architecture under radiographic guidance and controls. The hardening (polymerization) of the cement media or the mechanical interlocking of other biomaterials serve to buttress the bony vault of the vertebral body, providing both increased structural integrity and decreased potential for painful micromotion and progressive collapse of the vertebrae and spinal column.

Bone tamps (bone balloons or Kyphoplasty™), a contemporary balloon-assisted vertebroplasty alternative for treatment of VCF, and previously described, for example in U.S. Pat. Pub. No. 2005/0119662A1, also involves injection of a bone cement into a mechanically created bone void within vertebral body. In this alternative vertebroplasty procedure, a balloon tamp is first inserted into the structurally compromised vertebral body, often through a cannula. The bone balloon is then deployed under high pressure. The expanding balloon disrupts the cancellous bone architecture and physiological matrix circumferentially and directs the attendant bony debris and physiologic matrix toward the inner cortex of the vertebral body vault. The balloon tamp is then collapsed and removed, leaving a bony void or cavity. The remaining void or cavity is repaired by filling it with an appropriate biomaterial media, most often bone cement. In most cases, the treatment goals are to reduce or eliminate pain and the risk of progressive fracture of the vertebral body and its likely resulting morbidity, complications, and disability.

Although most of these interventional procedures are an improvement over previous conservative treatments that consisted of bed rest, pharmaceuticals, and/or cumbersome back braces, these methods still suffer from the complication of potential leakage of the therapeutic biomaterial repair media (bone cement, etc.) outside of the desired treatment zone. Numerous risks are associated with these spinal interventional procedures. The risks and complications, which are related to the leakage of the biomaterial into structures that are intended to be preserved, may involve extravasation of the biomaterial into veins and/or lungs, infections, bleeding, rib or pedicle fracture, pneumothorax, increased pain, a range of soft and/or neural tissue impingement, paresis, and paralysis. Most clinicians prefer to focus or contain treatments to the injured or diseased tissues alone.

Disease and injury also may erode or violate the supporting and collateral soft tissues. In the case of an insult, disruption, disease, or injury to a joint construct (spinal column [e.g., spinal facet], hip, knee, elbow, fingers, ankle, shoulder, synovium, collateral ligaments, etc.), joint capsule, ligamentous structures, or cartilaginous (collagen based) tissues, it may be necessary to manage or contain physiological biomaterial, or other therapeutic media within the joint or anatomic structure. Likewise, primary and secondary spinal tumors may contribute to a loss of tissue (bony, etc.) integrity and strength. Therefore, these tumors may serve as indications for vertebroplasty and other interventional spinal augmentation. The treatment of many other diseases of the bone and other tissues also may be facilitated by treating the diseases from within and/or proximate to the target anatomy. For example, chemotherapeutic agents may be implanted in proximity to or within a tumor. Or in the case of a failed bony fusion (pseudoarinrosis), a reoperation and revision may be avoided through the introduction of biological agents into a containment device designed to promote-bony healing. In particular, bone healing by interventional means may be facilitated by the implantation of osteophilic (osteoinductive or osteoconductive) materials, which are scaffolds and/or materials used to stimulate or optimize bony healing. These materials include, but are not limited to, hydroxylapaptite (HA), tri-calcium phosphate, biocoral, bioceramics, biomaterial granules, demineralized bone matrix (DBM), bone morphogenic proteins (BMPs), and collagen. Bone morphogenic proteins (BMPs), an active ingredient in DBM and a member of the TGF-β (transforming growth factorβ) super family, mediate developmental processes that include morphogenesis, differentiation, cell survival, and apoptosis. Although the role of TGF-β is not fully understood, its net effect is an increase in bone matrix. Other factors, such as insulin-like growth factors (IGF I and IGF II) and platelet derived growth factor are also important. Unfortunately, since these proteins have short biological half-lives, they must be maintained at the treatment zone in sufficient therapeutic concentrations in order to be effective. Therefore, dilution of the therapeutic agent due to the unintentional migration of the implanted material away from the therapeutic zone is also a major challenge to good patient outcomes.

Devices and methods for controlled delivery of therapeutic agents into bone and soft tissue were previously described in U.S. Pat. No. 6,960,215. These previously described containment devices were made of a fabric material.

It would be desirable to provide improved devices and methods that contain and deliver implanted biomaterial or other pharmacological or treatment media at any time during the treatment cycle, while preventing the unintentional migration of the implanted materials and/or controlling the release of the implanted materials into the targeted tissue or cellular treatment zone.

### SUMMARY

The invention comprises a containment device for site-specific delivery of therapeutic material without unintentional leakage into adjacent anatomy, comprising an expandable containment body comprising a naturally-derived collagenous material; and a delivery device to convey the containment body into a treatment location within a body, the delivery device having a proximal delivery device end, a distal delivery device end, and at least one lumen extending between the proximal delivery device end and the distal delivery device end. The containment body may be detachably attached to the distal end of the delivery device. The naturally-derived collagenous material may be submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa. The containment body may be perforated. The device may further comprise a guide sheath. The delivery device may be controllable from a proximal end of the guide sheath and movable between a first configuration for holding the expandable containment body in a radial compression having an unexpanded diameter, and a second configuration where the expandable containment body can assume an expanded diameter. The containment body may further comprise an opening through which the therapeutic material may be inserted. The opening of the containment body may be sealable. The delivery device may comprise a junction of interlocking ends that may be mechanically disconnectable. The therapeutic material may be integrated with the containment body prior to conveying the containment body into a treatment location within a body. Alternatively, the therapeutic material may be inserted following the delivery of the containment body. The therapeutic material may be a bone cement. The therapeutic material may be a biomaterial. The therapeutic material may be an antibiotic, a growth factor, or a chemotherapeutic agent. The delivery device may be a catheter.

The invention also comprises a method of treating a bone, an aneurysm, or arteriovenous malformations, comprising providing a containment device for site-specific delivery of therapeutic material.

The invention also comprises a containment device for site-specific delivery of a therapeutic material to a bony body without unintentional leakage into the adjacent anatomy, comprising an expandable containment body comprising a naturally-derived collagenous material; and a delivery device to convey the containment body into an interior of the bony body through an opening in a bony body, the delivery device having a proximal delivery device end, a distal delivery device end, and at least one lumen extending between the proximal delivery device end and the distal delivery device end. The containment body is detachably attached to the distal end of the delivery device. The naturally-derived coliagenous material may be selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.

The invention comprises a containment device for site-specific delivery of a therapeutic material to a vertebral body without unintentional leakage into the adjacent anatomy. The containment device comprises an expandable containment body comprising a naturally-derived collagenous material; and a delivery device to convey the containment body into an interior of the vertebral body, the delivery device having a proximal delivery device end, a distal delivery device end, and at least one lumen extending between the proximal delivery device end and the distal delivery device end. The containment body may be detachably attached to the distal end of the delivery device. The naturally-derived collagenous material is selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.

### BRIEF DESCRIPTION OF THE DRAWINGS

FIG. 1A is a lateral view of three normal vertebrae;

FIG. 1B is a lateral view of three vertebrae wherein the vertebral body of the middle vertebrae is compressed;

FIG. 2 is a lateral view of a compressed vertebra with bone cement extruded through the fractured vertebral vault;

FIG. 3A is a top view of a probe including a catheter tube with expandable structure in a substantially collapsed condition attached to the distal end of the catheter;

FIG. 3B is a schematic illustration of a probe, including a catheter tube with expandable containment structure comprising extracellular matrix (ECM) material in a expanded configuration attached to the distal end of the catheter;

FIG. 3C is a schematic illustration of a probe, including a catheter tube with expandable containment structure comprising ECM material in a substantially collapsed configuration attached to the distal end of the catheter in a guide sheath;

FIG. 4 is a lateral view of a transpedicular placement of a representative expandable containment device into a damaged vertebra;

FIG. 5 is a top view of a lumbar vertebra, partially cut away;

FIG. 6A is a lateral view of one posterior access route to the anterior vertebral body shown in FIG. 1; and

FIG. 6B is a top view of transpedicular and parapedicular routes to the anterior vertebral body.

### DETAILED DESCRIPTION OF THE DRAWINGS AND THE PRESENTLY PREFERRED EMBODIMENTS

The present invention relates to medical devices, and in particular, containment devices for implanting therapeutic materials in vivo. The containment device of the present invention is especially appropriate, but not limited to VCF treatments. For example, the containment device may be used for treatment of diverse organs and tissues, such as heart tissue and tissue located within the gastrointestinal tract and urological and gynecological systems; and blood vessels, including blood vessels in the brain. The containment device provides a barrier, preventing the unintentional migration of its augmentation, reconstructive, pharmacological, and therapeutic contents from the treatment site.

### Definition of Terms

The use of the terms “distal” and “proximal” are referenced from typically two different reference sources. The vascular medical community will typically reference a device from the heart. The rest of the medical community typically references “distal” and “proximal” with respect to the attending. In this Specification, “distal” means farthest from the physician, and “proximal” means closest to the physician.

The term “biocompatible material” refers to any material that is biologically compatible by not producing a toxic, injurious, or immunological response in living tissue. A large number of different types of biocompatible materials are known in the art which may be inserted within the body. Biocompatible materials include bioabsorbable materials that may be inserted within the body and that later dissipate. The term “bioabsorbable” is used herein to refer to materials selected to dissipate upon implantation within a body, independent of which mechanisms by which dissipation can occur, such as dissolution, degradation, absorption and excretion. The actual choice of which type of materials to use may readily be made by one ordinarily skilled in the art. Preferred biocompatible material is naturally-derived collagenous material.

The term “therapeutic material” refers to any material that may be used to fill in bone, body cavity, vasculature, or tissue sought to be treated. Therapeutic material includes, for example, bone cement, such as methylmethacrylate cement or a synthetic bone substitute that may be used to treat bone or bone cavity. Therapeutic materials also include, for example, a therapeutic agent, i.e., drug, or agents, or compositions comprising them. Examples of suitable therapeutic agents are provided below.

The terms “therapeutic agent” and “drug” are used interchangeably herein and include pharmaceutically active compounds, proteins, oligonucleotides, ribozymes, anti-sense genes, DNA compacting agents, gene/vector systems (i.e., anything that allows for the uptake and expression of nucleic acids), nucleic acids (including, for example, recombinant nucleic acids; naked DNA, cDNA, RNA; genomic DNA, cDNA or RNA in a non-infectious vector or in a viral vector which may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic polymers that may be selected from a number of types depending on the desired application.

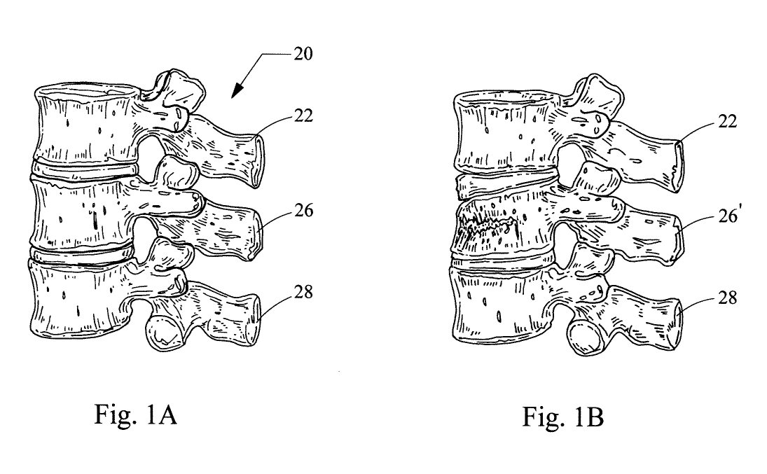

As shown in FIG. 1A, the lateral view of typical spinal motion segments 20 is depicted, with lumbar vertebrae 22, 26, and 28. In contrast, FIG. 1B illustrates a lateral view of a segment of a spinal column in which the middle vertebra 26′ is compressed. Compression can result from conditions such as osteoporotic fractures, malignant metastatic disease, and benign tumors of the bone and are suitable for treatment using the device of the present invention.

The percutaneous injection of bone cements, such as PMMA or the like, in vertebroplasty and kyphoplasty procedures has had some success in the treatment of pain associated with VCFs commonly found in osteoporosis patients. The bone cement is believed to solidify the porous inside and/or potential fractures on the outside of the vertebral body. When effectively injected, the bone cement is thought to prevent painful motion of the bony segments and to strengthen the spinal column to prevent further degradation and collapse. Leakage of the bone cement outside of the preferred treatment zone, however, not only does not alleviate the pain but also may lead to serious side effects.

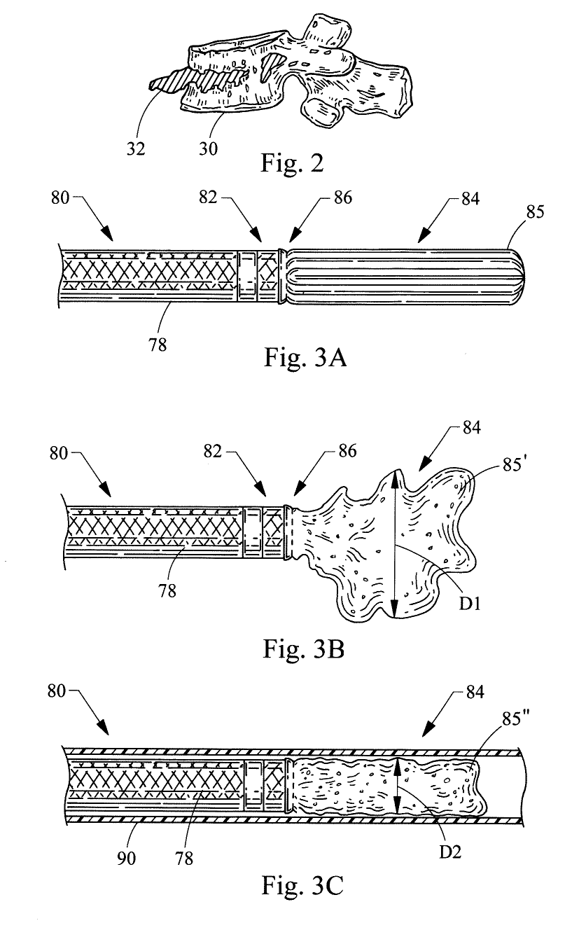

As shown in FIG. 2, where bone cement can extrude through the fractured vertebral vault 30, an exposed, sharp, abrasive, and durable surface 32 may be formed. This extruded media could erode nearby anatomic structures, causing further pain and complications. The precise direction, placement, and containment of therapeutic material and agents are fundamental to optimal patient outcomes. Latrogenic injury may be reduced or eliminated by the proper application of a containment technology. The containment device tends to prevent the unintentional migration of implanted therapeutic materials, such as bone cement, from the treatment site. The device, however, is not limited to the treatment of fractures in the vertebra. The containment device may be utilized in any other bone, or soft tissue, or vasculature where it is desired to control either the release or the unintentional migration of a therapeutic agent. For example, the containment device may be utilized to treat AVMs or aneurysms and/or may be used as an occluding device. Moreover, it may be utilized to concentrate therapeutic agents at the treatment site, resulting in their improved biomechanical function and/or therapeutic effect.

### Medical Device

The containment device may be a generally an expandable and/or fillable body that concentrates the focus of the therapeutic agent and reduces or prevents unintentional leakage or migration of therapeutic materials from the interior of the containment device into tissues or voids (i.e., adjacent anatomy) that are intended to be preserved. As depicted in FIGS. 3A-C, the device 84 may include an expandable body 85 (as in FIG. 3A), 85′ (as in FIG. 3B) and 85″ (as in FIG. 3C) made from a biocompatible material.

The body of the containment device may be formed of a variety of desirable, biocompatible implant materials suitable for bulking and supporting a target tissue, which do not interfere with the therapeutic material being delivered by the containment device. Materials that may be used include reconstituted or naturally-derived biocompatible materials, such as extracellular matrix (ECM) materials.

Preferred materials for use in this invention are reconstituted or naturally-derived collagenous materials isolated from suitable animal or human tissue sources. As used herein, it is within the definition of a “naturally-derived ECM” to clean, delaminate, and/or comminute the ECM, or to cross-link the collagen or other components within the ECM. It is also within the definition of naturally occurring ECM to fully or partially remove one or more components or subcomponents of the naturally occurring matrix.

Reconstituted or naturally-derived collagenous materials that are at least bioresorbable will provide advantage in the present invention, with materials that are bioremodelable and promote cellular invasion and ingrowth providing particular advantage.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials for use in the present invention may be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et aL., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et aL Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 pg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

Small intestine submucosa (hereinafter “SIS”) is one preferred form of ECM. SIS may be harvested and delaminated in accordance with the description in U.S. Pat. Nos. 4,956,178 and 4,902,508. SIS may be a preferred material because it has special bio-remodeling characteristics. Commercially available SIS material is derived from porcine small intestinal submucosa that remodels to the qualities of its host when implanted in human soft tissues. SIS is commercially available from Cook Biotech, West Lafayette, Ind.

SIS material may be in a form of a sponge-like or foam-like SIS (lyophilized SIS sponge, such as SURGISIS™ Soft-Tissue Graft (SIS) [Cook Biotech, Inc., West Lafayette, Ind.]) capable of greatly expanding in diameter as it absorbs therapeutic material, or non-sponge material comprising a sheet of SIS.

Although, reconstituted or naturally-derived collagenous materials are most preferred for use in this invention, the containment body may be formed from a variety of other biocompatible materials, including, for example, of biocompatible metals or other metallic materials; polymers including bioabsorbable or biostable polymers; stainless steels (e.g., 316, 316L or 304); nickel-titanium alloys including shape memory or superelastic types (e.g., nitinol or elastinite); noble metals including platinum, gold or palladium; refractory metals including tantalum, tungsten, molybdenum or rhenium; stainless steels alloyed with noble and/or refractory metals; silver; rhodium; inconel; iridium; niobium; titanium; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; magnetic ferrite; nonmetallic biocompatible materials including polyamides, polyolefins (e.g., polypropylene or polyethylene), nonabsorbable polyesters (e.g., polyethylene terephthalate) or bioabsorbable aliphatic polyesters (e.g., homopolymers or copolymers of lactic acid, glycolic acid, lactide, glycolide, para-dioxanone, trimethylene carbonate or ε-caprolactone); polymeric materials (e.g., poly-L-lactic acid, polycarbonate, polyethylene terephthalate or engineering plastics such as thermotropic liquid crystal polymers (LCPs)); biocompatible polymeric materials (e.g., cellulose acetate, cellulose nitrate, silicone, polyethylene terephthalate, polyurethane, polyamide, polyester, polyorthoester, polyanhydride, polyether sulfone, polycarbonate, polypropylene, high molecular weight polyethylene or polytetrafluoroethylene); degradable or biodegradable polymers, plastics, natural (e.g., animal, plant or microbial) or recombinant material (e.g., polylactic acid, polyglycolic acid, polyanhydride, polycaprolactone, polyhydroxybutyrate valerate, polydepsipeptides, nylon copolymides, conventional poly(amino acid) synthetic polymers, pseudo-poly(amino acids) or aliphatic polyesters (e.g., polyglycolic acid (PGA), polylactic acid (PLA), polyalkylene succinates, polyhydroxybutyrate (PHB), polybutylene diglycolate, poly epsilon-caprolactone (PCL), polydihydropyrans, polyphosphazenes, polyorthoesters, polycyanoacrylates, polyanhydrides, polyketals, polyacetals, poly(α-hydroxy-esters), poly(carbonates), poly(imino-carbonates), poly(β-hydroxy-esters) or polypeptides)); polyethylene terephthalate (e.g., dacron or mylar); expanded fluoropolymers (e.g., polytetrafluoroethylene (PTFE)); fluorinated ethylene propylene (FEP); copolymers of tetrafluoroethylene (TFE) and per fluoro(propyl vinyl ether) (PFA)); homopolymers of polychlorotrifluoroethylene (PCTFE) and copolymers with TFE; ethylene-chlorotrifluoroethylene (ECTFE); copolymers of ethylene-tetrafluoroethylene (ETFE); polyvinylidene fluoride (PVDF); polyvinyfluoride (PVF); polyaramids (e.g., kevlar); polyfluorocarbons including polytetrafluoroethylene with and without copolymerized hexafluoropropylene (e.g., teflon or goretex); expanded fluorocarbon polymers; polyglycolides; polylactides; polyglycerol sebacate; polyethylene oxide; polybutylene terepthalate; polydioxanones; proteoglycans; glycosaminoglycans; poly(alkylene oxalates); polyalkanotes; polyamides; polyaspartimic acid; polyglutarunic acid polymer; poly-p-diaxanone (e.g., PDS); polyphosphazene; polyurethane including porous or nonporous polyurethanes; poly(glycolide-trimethylene carbonate); terpolymer (copolymers of glycolide, lactide or dimethyltrimethylene carbonate); polyhydroxyalkanoates (PHA); polyhydroxybutyrate (PHB) or poly(hydroxybutyrate-co-valerate) (PHB-co-HV); poly(epsilon-caprolactone) (e.g., lactide or glycolide); poly(epsilon-caprolactone-dimethyltrimethylene carbonate); polyglycolic acid (PGA); poly-L and poly-D(lactic acid) (e.g., calcium phosphate glass); lactic acid/ethylene glycol copolymers; polyarylates (L-tyrosine-derived) or free acid polyarylates; polycarbonates (tyrosine or L-tyrosine-derived); poly(ester-amides); poly(propylene fumarate-co-ethylene glycol) copolymer (e.g., fumarate anhydrides); polyanhydride esters; polyanhydrides; polyorthoesters; prolastin or silk-elastin polymers (SELP); calcium phosphate (bioglass); compositions of PLA, PCL, PGA ester; polyphosphazenes; polyamino acids; polysaccharides; polyhydroxyalkanoate polymers; various plastic materials; teflon; nylon; block polymers or copolymers; Leica RM2165; Leica RM2155; organic fabrics; biologic agents (e.g., protein, extracellular matrix component, collagen, fibrin); collagen or collagen matrices with growth modulators; aliginate; cellulose and ester; dextran; elastin; fibrin; gelatin; hyaluronic acid; hydroxyapatite; polypeptides; proteins; ceramics (e.g., silicon nitride, silicon carbide, zirconia or alumina); bioactive silica-based materials; carbon or carbon fiber; cotton; silk; spider silk; chitin; chitosan (NOCC or NOOC-G); urethanes; glass; silica; sapphire; composites; any mixture, blend, alloy, copolymer or combination of any of these; or various other materials not limited by these examples.

In general, preferred materials for use in this invention will biodegrade in vivo in a matter of months, although some more crystalline forms may biodegrade more slowly.

In certain embodiments, the containment material may be porous, semi-porous, or non-porous. It may be made from a continuous material with uniform properties or it may be interrupted or fenestrated to achieve the treatment objectives. In some instances, the materials may have a variable thickness or durometer (hardness) to achieve specialized geometric deployment.

The containment device may be of many different shapes and forms depending on the tissue to be treated and the intended therapeutic effect. In many instances, where the wall of the containment device is made from a relatively soft, flexible material, such as a fabric or a membrane, the containment device could conform to the cavity inside of the vertebral or other bony body or soft tissue being treated. Alternatively, when the device is made of a semi-compliant or rigid material, the containment device could have a pre-determined shape. Preferably, the containment body is made from a relatively soft and flexible material, such as ECM.

For example, the device may serve a directional or containment function by directing, channeling, or concentrating the treatment media within a specific anatomic orientation or structure or into a target treatment area. In such a case, the device may be closed like a “pouch” that may be sealed after filling, open like a “stent” to channel the material more precisely, or perforated like a sponge or foam to contain the material within. A pouch, sponge-like or foam-like configurations for the containment device are preferred.

Similarly to stent, the containment device may self-expand and assume the geometry of a curved column (similar to a sausage casing or linked sausage casings), with either a closed or open end, that could serve to capture and/or channel the therapeutic media to achieve an optimal medical outcome.

For example, the physician could carve out a curved void in the anterior region of the vertebral body and then deploy the elongated, curved device into the cavity. Where the device has at least one open end, e.g., similar to a curved hollow tube, the therapeutic media would leak out the open ends of the device, coming into contact with the cancellous bone along the lateral edges of the verteberal body. Although the therapeutic media, e.g., bone cement, would subsequently invade the interstices of the cancellous bone, the containment channel would still serve its intended purpose by preventing the bone cement from entering the venous plexus. Where the device has only one open end for delivery of the therapeutic material, the material would be contained within the device.

The containment device may also be made from a sheath of material to have a bulbous geometry that may be manipulated to assume alternative shapes as it conforms to the anatomy where it is inserted. During or after deployment of the device, application of an external force could cause the containment device to deform plastically into the shape or space of the tissues that are to be treated. For example, after filling with the therapeutic material, the containment device may be collapsed to assume a concave disk-like geometry or other geometry.

As shown in FIG. 3B, the containment device 84 may have a sponge-like or a foam-like form and geometry that also may be manipulated to assume shape of the anatomy where it is inserted. This is because following the deployment of the device 84, the injection of therapeutic material will cause the expandable body 85 of the device to “swell” to a second configuration 85′ where the device 84 may assume an expanded diameter D1, and fill in the space by conforming to the shape or space of the anatomy to be treated. As illustrated in FIG. 3C, for delivering the containment device 84, the device may be radially compressed to an unexpanded diameter D2 of expandable body 85″ in a guide sheath 90.

The device may be a double (or multiple nested) containment device where there are at least two devices nested within each other. For example, one containment device would surround the other and each would be capable of being filled with a therapeutic material. In the treatment of a soft tissue lesion (e.g., tumor, etc.), it may be beneficial to have an inner containment device with a structural material to provide load-bearing support, while filling the outer containment device with a chemotherapeutic agent. In this manner, as the lesion responds to chemotherapeutic agent and “shrinks,” the structural material could remain intact to support the tissue that remains.

Numerous delivery devices may be used in conjunction with the containment device, enabling the placement of the containment device in the proper treatment site. These include, but are not limited to, a catheter, cannula, needle, syringe, or other expandable delivery device. For example, as shown in FIGS. 3A-3C, a delivery device may include a catheter 78 having a proximal end 80 and a distal end 82. A proximal end 86 of a containment device 84 is attached to the distal end 82 of the catheter 78 in an appropriate manner, e.g., cyanoacrylate glue (or other appropriate adhesive) or construct welded joints (metallic and non-metallic), that may best serve any desirable detachment system. These detachment systems include any joint severable by electrolytic, mechanical, hydraulic, photolytic, thermal, or chemical means.

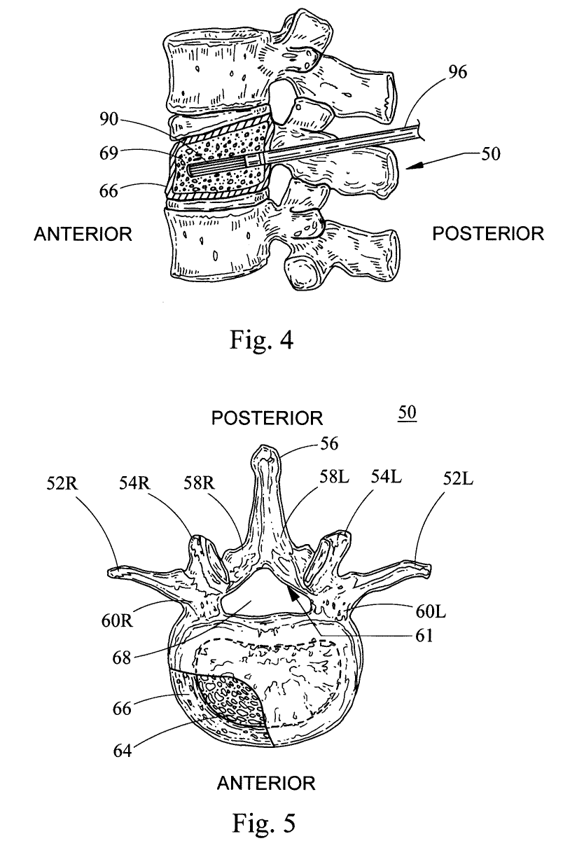

Depending on the patient's condition, the physician may choose to modify or accessorize the containment device as needed. For example, the device may be permanently or temporarily implanted. Referring to FIGS. 4 and 5, the device 90 may be inserted through a hole 69 in the cortical bone 66 of a lumbar vertebra 50. Where the device 90 is to be implanted in the patient permanently, various detachment technologies may be employed after the containment device and therapeutic material is delivered to the proper treatment site.

Additional detachment means are known in the art and may include, but are not limited to, electrolytic detachment; mechanical interference fit (Morse-taper-type, and the like) that may be detached by hydraulic technologies, ball valves, gas pressure changes; breakaway designs (severable by force or exposure to an alternate internal or external technology); photolytic means (severable by exposure to light, laser, and the like); thermal modulation (heat, cold, and radio frequency); mechanical means (screwing/unscrewing); and bioresorbable technologies (severable by exposure to an aqueous solution such as water, saline, and the like).

Further detachment means, such as thermal, photolytic, and via severable junction were described in detail in U.S. Pat. No. 6,960,215, disclosure of which is incorporated herein in its entirety.

Further, the containment device 90 may be detached through mechanical means. This could include various designs of interlocking ends that are held together by a sleeve. Different types of mechanically deployable joints that may be adapted for use with the containment device 90 are described in U.S. Pat. Nos. 5,234,437; 5,250,071; 5,261,916; 5,304,195; 5,312,415; and 5,350,397, the entirety of which are herein expressly incorporated by reference.

Many different methods may be used to seal the containment device, if necessary. For example, the containment device may contain a self-sealing one-way valve, or a plug, such as a detachable silicone balloon, may be used to seal the neck of the containment device. The containment device also may adhere to itself where it is made from a material with appropriate adhesive and/or elastic properties, thereby sealing the contents inside. Suitable sealants and sealant method are known to those who are skilled in the art may be employed to close the containment device and prevent the unintentional migration of its contents from the treatment site.

Where the device or a portion of the device is only intended to be implanted temporarily, the device may be collapsed and subsequently removed from the body after the contents of the containment device have substantially migrated outside of the device or when it is desired. In order to facilitate navigation, detachment, removal, and implantation of the containment device, all or portions of the surfaces of the access, delivery, and containment devices may be modified.

Surface modifications and methods may include, but not be limited to, ion bombardment, physical vapor deposition plasma coatings, water-soluble neuroprotectant or vascular protectant coatings (heparin, etc.), hydrophilic coatings, anti-adhesion coatings, peptide coatings, gene therapy treatments, anti-corrosion coatings, electrically insulating coatings, or other technologies as known in the art. These coatings may prevent further injury to the patient while the device is being removed since the coating may decrease the risk of scar tissue forming around the implanted foreign devices. As is well-known to one skilled in the art, any number of surface modifications may complement the utility of the device applications and outcomes. In addition, retrievable containment devices may utilize different delivery systems than those used in the case of detachable devices. In particular, catheters capable of electrolytic detachment may not be chosen in order to avoid the possibility of accidental detachment due to unintentional exposure of the electrolytic joint to an ionic environment.

Additional substances that enhance the delivery and therapeutic effect of the therapeutic materials also may be impregnated or otherwise incorporated into the containment device. These include, but are not limited to, hydrogels, hydrophilic coatings, anti-adhesion media, peptides, and genes.

### Therapeutic Materials and Agents

A wide range of materials may be placed or incorporated into or coated onto the outside of the containment device prior to the delivery of the device. Alternatively, therapeutic materials also may be used to fill the containment device following the delivery and deployment of the containment device. For example, bone cement, such as PMMA or the like, may be injected into the containment device to treat compression fractures in the vertebral bodies. Likewise, any number of polymer or liquid formulas, properly contained or channeled, may serve the therapeutic requirements equally well. The biomaterial need only be adapted to physiology, which is primarily its viscoelastic and strength requirements, suited to the fit, form, and function of the treated structures and clinical outcome requirements.

Where the wall of the containment device is formed from a non-porous material, the device may prevent the material, e.g., PMMA or epoxy, from “leaking” outside of the vertebra. In the alternative, if the wall is formed from a porous material, the implanted materials may migrate or diffuse away from the containment device into the surrounding area. For example, where the containment device contains large pores, it may be filled with bone cement, such as PMMA or the like, possibly under pressure, until the device reaches its maximum capacity. The bone cement may then begin to seep out of the pores to form protrusions in the form of bumps or rods of bone cement extruding in an unorganized manner from the containment device. In addition to filling any remaining voids in the cancellous bone of the vertebral body, the extruded spikes may aid in anchoring the containment device in its proper therapeutic place, even if the vertebral body later changes shape due to further deterioration. In light of the progressive deterioration of bones seen in diseases such as osteoporosis and cancer, these extruded rods could provide much needed continued support even after the bone resorbs.

In addition, one therapeutic material, such as bone cement may carry another therapeutic material, such as a drug. Alternatively, the drug may be separately delivered before injection of the bone cement. Thus, using an expandable containment body, the physician is able to treat a fracture while also delivering a desired therapeutic substance (like an antibiotic, bone growth facer or osteoporosis drug) to the site.

Where the containment device is made from porous or semi-porous materials, the therapeutic materials may escape or diffuse through the pores into the surrounding environment. The appropriate degree of porosity or permeability may be determined in order to achieve the correct dosing and may depend in part on the concentration of the therapeutic agent and the size of the treatment site. Similarly, the containment device may serve as a time-release or dosing vessel in delivering the therapeutic material, such as drug, where a bio-resorbable material, such as collagenous material or poly-lactic acid (PLA), is used. In the treatment of fractures, osteoconductive materials, which provide scaffolds on which new bones can grow, and osteoinductive materials, which activate stem cells to promote and/or induce bone formation, would be useful in treating compression fractures and enhancing bone growth.

Alternatively, therapeutic materials may be placed or incorporated into, or coated onto the containment device. For example, therapeutic material may be incorporated into the material that makes up the containment device prior to the delivery of the containment device by dipping the device in a medical formulation (often a dry powder, liquid or gel) containing a medically-effective amount of any desired therapeutic material, such as antibiotic, bone growth factor or other therapeutic agent to coat the body with the above-mentioned substance before it is inserted into a location being treated.

Optionally, the containment body may be wholly or partially expanded before the coating is performed. Optionally, the coated containment body may be dried with air or by other means when the applied formulation is wet, such as a liquid or a gel. The containment device may be refolded as required and either used immediately or stored, if appropriate and desired. Coated on the containment device, therapeutic substances may be delivered to the desired location requiring treatment.

Methods of coating medical devices with therapeutic substances are known in the art.

To deliver the therapeutic material, the containment device may be impregnated with the therapeutic material. For example, to impregnate the device with the therapeutic material, therapeutic material may be pre-mixed with a comminuted ECM material during the preparation of the ECM material.

Possible therapeutic materials to be delivered via the containment device include, but are not limited to, bone cements and other autogenous tissues or cells, donor tissues or cells, bone substitutes, bone morphogenic proteins (e.g., BMP-2 or OP-1), growth factors (e.g., TGF-β, IGF I, IGF II, and platelet-derived growth factor), tissue sealants, chemotherapeutic agents, and other pharmaceutical agents.

Examples of antibiotics that may be used to treat bone infection include, for example, gentamicin, ancef, nafcillin, erythromycin, tobramycin, and gentamicin. Typical bone growth factors are members of the Bone Morphogenetic Factor, Osteogenic Protein, Fibroblast Growth Factor, Insulin-Like Growth Factor and Transforming Growth Factor alpha and beta families. Chemotherapeutic and related agents include compounds such as cisolatin, doxcrubicin, daunorubicin, methotrexate, taxol and tamoxifen. Osteoporosis drugs include estrogen, calcitonin, diphosphonates, and parathyroid hormone antagonists.

In addition to the agents listed above in connection with treatment of bone related conditions, the following therapeutic agents also may be used with the containment device of this invention to treat various other conditions: antiproliferative/antimitotic agents including natural products such as vinca alkaloids (i.e. vinblastine, vincristine, and vinorelbine), paclitaxel, epidipodophyllotoxins (i.e. etoposide, teniposide); antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as (GP) II _{b}/IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives, i.e., aspirin; para-aminophenol derivatives, i.e., acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat) and protease inhibitors, mixtures and compositions comprising them.

Medically-effective amounts of therapeutic substances are defined by their manufacturers or sponsors and are generally in the range of 10 nanograms to 50 milligrams per site, although more or less may be required in a specific case. For example, a containment device for use in a bone can accommodate a typical dose of the antibiotic, gentamicin, to treat a local osteomyelitis (bone infection). A typical dose is about 1 gram, although the therapeutic range for gentamicin is far greater, from 1 nanogram to 100 grams, depending on the condition being treated and the size of the area to be covered. A medically-suitable gel formulated with appropriate gel materials, such as Polyethylene glycol, can contain 1 gram of gentamicin in a set volume of gel, such as 10 cc. Not only can the dose be optimized, but additional doses may be applied at later times without open surgery, enhancing the therapeutic outcome.

The therapeutic material may be combined with a pharmaceutically acceptable carrier. The term “pharmaceutically acceptable carrier” includes any material which, when combined with a therapeutic material, allows the specific therapeutic agent to retain biological activity and is non-reactive with the subject's immune system. Examples of pharmaceutically acceptable carriers include, but are not limited to, any of the standard pharmaceutical carriers such as a phosphate buffered saline solution, water, emulsions such as oil/water emulsions, various polymer carrier materials, and various types of wetting agents. Compositions comprising such carriers are formulated by well known conventional methods (see, for example, Remington's Pharmaceutical Sciences, Chapter 43, 14th Ed., Mack Publishing Co., Easton, Pa.).

### Methods of Use of Containment Device

As noted above, use of the containment device is not limited to treatment of vertebral ailments. However, such procedures are discussed here for exemplary purposes. Before discussing such methods of operation, various portions of the vertebra are briefly discussed.

FIG. 5 depicts a top view of a vertebra 50. At the posterior of the vertebra are a right and left transverse process 52R, 52L, a right and left superior articular process 54R, 54L, and a spinous process 56. The right and left lamina, 58R, 58L, lie in between the spinous process 56 and the superior articular processes 54R, 54L, respectively. A right and left pedicle, 60R; 60L, are positioned anterior to the right and left transverse process, 52R, 52L. A vertebral arch 61 extends between the pedicles 60 and through the lamina 58. A vertebral body is located at the anterior of the vertebra 50 and joins the vertebral arch 61 at the pedicles 60. The vertebral body includes an interior volume of reticulated, cancellous bone 64 enclosed by a compact, cortical bone 66 around the exterior. The vertebral arch 61 and body make up the spinal canal, i.e., the vertebral foramen 68; the opening through which the spinal cord and epidural veins pass.

Referring back to FIG. 4, the device may include a detachable containment device 90 mounted on a delivery device 96 that is used to position, deploy, and fill the containment device 90. The physician can choose from a variety of approaches to insert the containment device into the vertebral body.

For example, the method may include gaining access to the interior of the vertebral body through a naturally occurring bore or passage in the vertebra (not shown) formed as a result of the condition to be treated. Alternatively, a bore or passage in the bone may be formed with a drill. Preferably, the size of the bore or passage into the interior of the vertebral body should be slightly larger than the external diameter of the implant body in its relaxed or pre-deployed state so that the containment device may be inserted through the bore into the vertebral body. In addition, the physician may further create a cavity 69 within the vertebral body before insertion of the device 90 if desired (as shown in FIG. 4). This may be accomplished using any surgical tool to carve out a cavity or perhaps by using an additional expandable or deployable device, such as those used in angioplasty or atraumatic tissue expansion or dissection. The containment device is preferably placed in the center of the vertebral body void or vault in order to distribute support evenly to the entire structure and to the physiological loads typical a living organism.

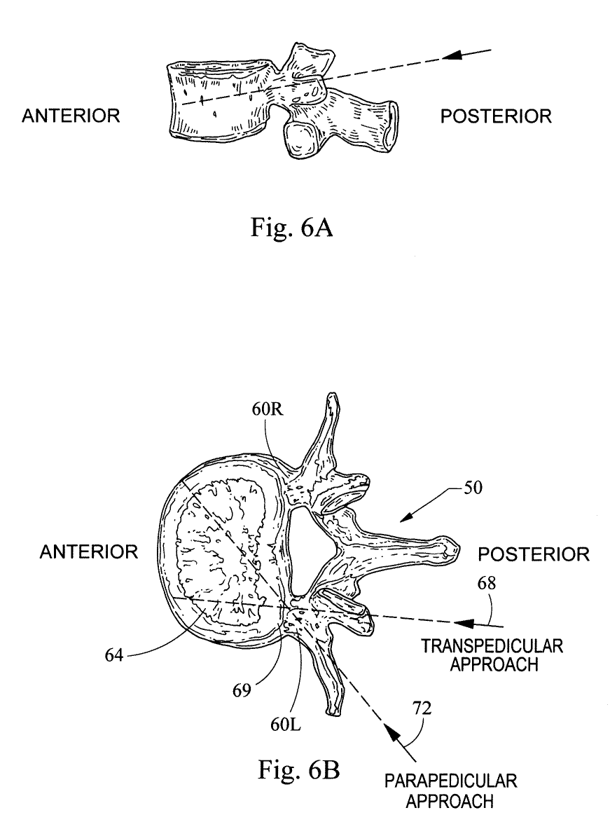

As shown in FIGS. 6A and 6B, a transpedicular approach 68 or parapedicular approach 72 may be used to gain access to the cancellous bone 64 in the vertebral body 62 through the pedicle 60 or through the side of the vertebral body beside the pedicle, respectively. The parapedicular approach 72 may especially be chosen if the compression fracture has resulted in collapse of the vertebral body below the plane of the pedicle. Still other physicians may opt for an intercostal approach through the ribs (not shown) or a more clinically challenging anterior approach (not shown) to the vertebral body.

As previously described, the containment device may be delivered to the treatment site using many different delivery devices including, but not limited to, a catheter, cannula, needle, syringe, or other delivery device. For example, the containment device 90 may be delivered to the treatment site via a guide sheath (not shown) through which a delivery device, such as a braided catheter 96 with the attached flexible containment device 90 in a substantially radially collapsed condition having an unexpanded diameter, may be pushed through the guide sheath to the interior of the bony body. The guide sheath may be combined with an obturator, access needle, or the like, and tunneled through intervening tissue to gain access to the treatment site. Once at the treatment site the containment device assumes the expanded diameter. The guide sheath may then be retracted towards its proximal end, thereby releasing the device 90 into the interior of the vertebral body or other treatment site. Many delivery devices and methods may be employed to deliver the containment device to the treatment site and are well known to those who are skilled in the art.

The device 90 may be deployed using any appropriate mechanical mechanism. This mechanical mechanism may be such that the containment device 90 may displace portions of the cancellous bone within the vertebral body upon deployment to create a cavity before it is filled with therapeutic materials. Alternatively, the device 90 may be filled directly with the therapeutic agent, possibly under pressure. For example, material such as collagenous material making up the containment device will absorb the therapeutic material causing the collagenous material to expand.

Where the containment device is self-expanding, similar to a stent, upon release from the guide sheath, the containment device may assume its primary shape within the cavity or void in which it is placed without the aid of any external forces. The device could subsequently be filled with the desired therapeutic material.

Alternatively, the original shape of the device may be manipulated into another secondary shape with the application of an external force. For example, the containment device may be constrained in its substantially radially collapsed condition within the inner lumen of the braided microcatheter until final anatomic positioning is achieved. A pusher wire may then be advanced, pushing the containment device outside of the braided delivery catheter. The device may be shaped into other geometries appropriate to the anatomy to be treated, geometries that would also improve the acceptance of therapeutic material and ultimately improve the therapeutic outcome.

Other ailments, which are not specific to bone, also may be treated with the present invention. For example, in the case of cancer, whether it be in the bone or soft tissue, placement of a containment device into or near the tumor could allow for the delivery of chemotherapeutic agents directly to the tumor. Where the containment device is made from porous, semi-porous, or bio-resorbable material, such as ECM, the chemotherapeutic agents contained within the containment device may be able to diffuse to the surrounding area. The containment device may be placed inside of a tumor using an appropriate interventional technique. For example, a guide sheath may be used to tunnel through adjacent tissue. The containment device may then be inserted into the desired therapeutic site through the guide sheath. When necessary, the containment device may be attached to the soft tissue. Sutures, or other methods that are well known to those who are skilled in the art, may be used to stabilize the placement of the containment device. In the case of deep wounds, the containment device may be used to deliver antibodies to the site.

Myofascial pain syndrome, which is a condition of the tissues characterized by intense localized pain coming from muscles and their respective connective tissues, also may be treated. A containment device made from porous, semi-porous, or bio-resorbable material may be placed in between the muscle fascia, providing for the controlled release of muscle relaxants and other therapeutic agents that may help to treat the syndrome as the therapeutic agents diffuse away from the containment device.

Plantar Fasciitis, which is an inflammation of the plantar fascia tissue at its attachment to the heel bone, also may be treated through placement of the containment device near the plantar fascia (a tough, fibrous band of connective tissue that extends over the sole of the foot). Similar to the above examples, the containment device may provide for the controlled delivery of anti-inflammatory drugs and other therapeutic agents that may provide relief from the acute pain associated with the condition.

The containment device also may be used for treatment of aneurysm, such as aortic abdominal aneurysm (AAA). An aneurysm is an area of a localized widening (dilation) of a blood vessel. The containment device may be placed in the blood vessel at the location of the aneurysm and provide for controlled delivery of therapeutic material while also providing a structural support for the dilated blood vessel.

Arteriovenous malformations (AVM), which are groups of abnormal vessels which may occur within the brain and other parts of the body, also may be treated through placement of the containment device within or near the abnormal vessel. AVM's develop when there are abnormal communications that directly connect relatively large arteries to veins; thus, the blood is exchanged at a relatively higher pressure with more rapid flow directly into the veins. This unusual connection between arteries and veins is called a nidus. The anatomy of the vein is not designed to take the higher pressures and flow; thus, it expands and pushes against the normal brain tissue. This may damage the normal brain causing weakness, numbness, loss of vision, or seizures. Often there is a rupture in the supplying arteries, the AVM itself, or the enlarged veins which results in an intracranial hemorrhage, which is a type of stroke. The containment device also may be used as an occluding device.

It is therefore intended that the foregoing detailed description be regarded as illustrative rather than limiting, and that it be understood that it is the following claims, including all equivalents, that are intended to define the spirit and scope of this invention.

1. A containment device for site-specific delivery of a therapeutic material without unintentional leakage into adjacent anatomy, comprising
a. an expandable containment body comprising a naturally-derived collagenous material; and
b. a delivery device to convey the containment body into a treatment location within a body, the delivery device having a proximal delivery device end, a distal delivery device end, and at least one lumen extending between the proximal delivery device end and the distal delivery device end,
wherein the containment body is detachably attached to the distal end of the delivery device.

2. The device of claim 1, wherein the naturally-derived collagenous material is selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.

3. The device of claim 1, wherein the containment body is perforated.

4. The device of claim 1, wherein the containment body further comprises the therapeutic material.

5. The device of claim 4, wherein the therapeutic material is integrated with the containment body prior to conveying the containment body into a treatment location within a body.

6. The device of claim 1, wherein the therapeutic material is inserted following the delivery of the containment body.

7. The device of claim 1, further comprising a guide sheath.

8. The device of claim 7, wherein the delivery device is controllable from a proximal end of the guide sheath and movable between a first configuration for holding the expandable containment body in a radial compression having an unexpanded diameter, and a second configuration where the expandable containment body can assume an expanded diameter.

9. The device of claim 1, wherein the containment body further comprises an opening through which the therapeutic material may be inserted.

10. The device of claim 9, wherein the opening of the containment body is sealable.

11. The device of claim 1, wherein the therapeutic material is a bone cement.

12. The device of claim 1, wherein the therapeutic material is a biomaterial.

13. The device of claim 1, wherein the therapeutic material is an antibiotic, a growth factor, or a chemotherapeutic agent.

14. The device of claim 1, wherein the delivery device is a catheter.

15. A method of treating a bone, an aneurysm, or arteriovenous malformations, comprising providing the containment device of claim 1.

16. The method of claim 15, wherein the naturally-derived collagenous material is selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.

17. A containment device for site-specific delivery of a therapeutic material to a bony body without unintentional leakage into the adjacent anatomy, comprising:
a. an expandable containment body comprising a naturally-derived collagenous material; and
b. a delivery device to convey the containment body into an interior of the bony body through an opening in a bony body, the delivery device having a proximal delivery device end, a distal delivery device end, and at least one lumen extending between the proximal delivery device end and the distal delivery device end,
wherein the containment body is detachably attached to the distal end of the delivery device.

18. The device of claim 17, wherein the naturally-derived collagenous material is selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.

19. A containment device for site-specific delivery of a therapeutic material to a vertebral body without unintentional leakage into the adjacent anatomy, comprising
a. an expandable containment body comprising a naturally-derived collagenous material;
b. a delivery device to convey the containment body into an interior of the vertebral body, the delivery device having a proximal delivery device end, a distal delivery device end, and at least one lumen extending between the proximal delivery device end and the distal delivery device end,
wherein the containment body is detachably attached to the distal end of the delivery device.

20. The device of claim 19, wherein the naturally-derived collagenous material is selected from the group consisting of submucosa, renal capsule membrane, dermal collagen, dura mater, pericardium, fascia lata, serosa, peritoneum or basement membrane layers, liver basement membrane, intestinal submucosa, small intestinal submucosa, stomach submucosa, urinary bladder submucosa, and uterine submucosa.
